(19) United States
(12) Patent Application Publication
(10) Pub. No.: US 2007/0015832 A1
(43) Pub. Date: Jan. 18, 2007

(54) Methods of treating overactive bladder and urinary incontinence

(75) Inventor: Mark Laughlin, Sandy, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/487,177

(22) Filed: Jul. 14, 2006

Related U.S. Application Data
(60) Provisional application No. 60/699,727, filed on Jul. 14, 2005.

(51) Int. Cl. A61K 31/192; A61K 31/41; A61K 31/381; A61K 31/35

(52) U.S. Cl. 514/568; 514/438; 514/460; 514/381

(57) ABSTRACT

The invention relates to methods of treating or slowing the onset of overactive bladder or urinary incontinence, or a symptom thereof selected from urinary frequency, urinary urgency, nocturia, or enuresis comprising identifying and administering to a subject in need of treatment a therapeutically effective amount of a compound according to Formulae I-V, as defined herein.

### CROSS REFERENCE TO RELATED U.S. APPLICATION

This application claims the benefit of U.S. Provisional Application Ser. No. 60/699,727, filed on Jul. 14, 2005, which is incorporated herein by reference in its entirety.

### FIELD OF THE INVENTION

The invention relates to therapeutic compounds and to the use of such compounds for treating overactive bladder and urinary incontinence.

### BACKGROUND OF THE INVENTION

Overactive bladder and urinary incontinence are medical conditions affecting at least 13 million Americans. Symptoms of overactive bladder and urinary incontinence include urinary frequency, urinary urgency, nocturia, and enuresis. Although the exact cause of overactive bladder and urinary incontinence are unknown, the disorder may result from hypersensitivity or destruction of sensory neurons of the bladder. For example, overactivity and/or instability of the detrusor muscles, which is mediated by muscarinic receptors in the bladder, is a major physiological effect typically found in overactive bladder and urinary incontinence patients.

Current treatments for overactive bladder and urinary incontinence include behavioral modification, devices, surgery, and medications. The primary medications for treating such disorders are antimuscarinics (which are members of the general class of anticholinergics). However, treatment with antimuscarinics suffers from limited efficacy and side effects such as dry mouth, dry eyes, dry vagina, blurred vision, drowsiness, urinary retention, weight gain, hypertension, constipation, and cardiac side effects, such as palpitation and arrhythmia, which are difficult for some individuals to tolerate. Accordingly, there is a need for new therapies and treatments for overactive bladder and urinary incontinence.

### BRIEF SUMMARY OF THE INVENTION

The invention relates to methods of treating or slowing the onset of overactive bladder and urinary incontinence, or a symptom thereof selected from urinary frequency, urinary urgency, nocturia, or enuresis. In one aspect, the method comprises identifying and administering to a subject in need of treatment a therapeutically effective amount of an Aβ₄₂ lowering agent, such as a compound according to Formulae I-V: 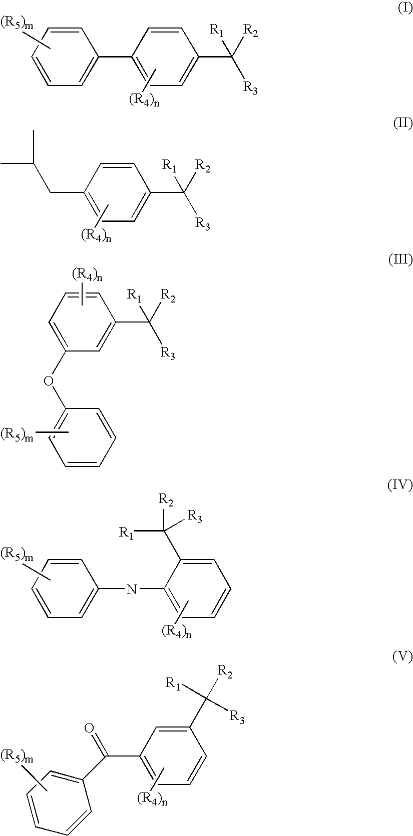
or pharmaceutically acceptable salts or solvates thereof, wherein:

R₁ is chosen from —CH₃, —CH₂CH₃, —CH₂CH₂CH₃, and —CH₂CH₂CH₂CH₃ (or can be taken together with R₂ to give a cyclopropyl ring, a cyclobutyl ring, a cyclopentyl ring, or a cyclohexyl ring);

R₂ is chosen from —CH₃, —CH₂CH₃, —CH₂CH₂CH₃, and —CH₂CH₂CH₂CH₃, be taken together with R₁ to give a cyclopropyl ring, a cyclobutyl ring, a cyclopentyl ring, or a cyclohexyl ring);

R₃ is chosen from —COOH, —COOR₆, —CONH₂, —CONHR₆, —CONR₆R₇, —CONHSO₂R₆, tetrazolyl, and a —COOH bioisostere;

R₄ is chosen from —Cl, —F, —Br, —I, —CF₃, —OCF₃, —SCF₃, —OCH₃, —OCH₂CH₃, —CN, —CH═CH₂, —CH₂OH, and —NO₂;

R₅ is chosen from —Cl, —F, —Br, —I, —CF₃, —OCF₃, —SCF₃, —OCH₃, —OCH₂CH₃, —CN, —CH═CH₂, —CH₂OH, and —NO₂;

R₆ is chosen from —CH₃, —CH₂CH₃, —CH₂CH₂CH₃, and —CH₂CH₂CH₂CH₃;

R₇ is chosen from —CH₃, —CH₂CH₃, —CH₂CH₂CH₃, and —CH₂CH₂CH₂CH₃;

m is an integer chosen from 0, 1, 2, and 3; and

n is an integer chosen from 0, 1, 2, and 3.

In specific embodiments, the compound is (R)-2-(2-fluoro-4-biphenyl)propionic acid. In certain embodiments, overactive bladder is treated. In other embodiments urinary incontinence is treated. In additional embodiments, symptoms of overactive bladder and urinary incontinence selected form urinary frequency, urinary urgency, nocturia, and enuresis are treated.

In another aspect of the invention, compositions and methods are provided for treating or slowing the onset of overactive bladder and urinary incontinence, or symptoms thereof, comprising identifying and administering to a subject in need a therapeutically effective amount of an Aβ₄₂ lowering agent, such as a compound according to Formulae I-V, in combination with at least one additional therapeutic agent.

In another aspect of the invention, compositions and methods are provided for treating or slowing the onset of overactive bladder and urinary incontinence, or symptoms thereof, comprising identifying and administering to a subject with Alzheimer's disease who is in need such treatment a therapeutically effective amount of an Aβ₄₂ lowering agent, such as a compound according to Formulae I-V. Such subject may be administered an Aβ₄₂ lowering agent, in combination with at least one additional therapeutic agent. In specific embodiments, the Aβ₄₂ lowering agent, is (R)-2-(2-fluoro-4-biphenyl)propionic acid. In certain embodiments, overactive bladder is treated. In other embodiments urinary incontinence is treated. In additional embodiments, symptoms of overactive bladder and urinary incontinence selected form urinary frequency, urinary urgency, nocturia, and enuresis are treated.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

The invention relates to methods of treating or slowing the onset of overactive bladder and urinary incontinence, or a symptom thereof selected from urinary frequency, urinary urgency, nocturia, or enuresis. In one aspect, the method comprises identifying and administering to a subject in need of treatment a therapeutically effective amount of an Aβ₄₂ lowering agent, such as a compound according to Formulae I-V: 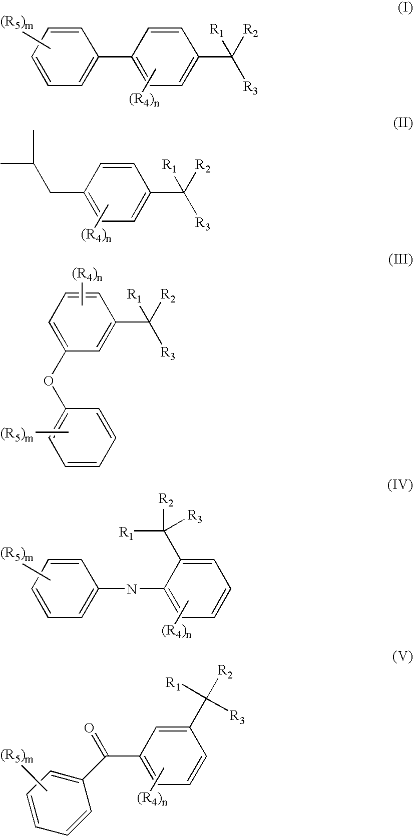
or pharmaceutically acceptable salts or solvates thereof, wherein:

R₁ is chosen from —CH₃, —CH₂CH₃, —CH₂CH₂CH₃, and —CH₂CH₂CH₂CH₃ (or can be taken together with R₂ to give a cyclopropyl ring, a cyclobutyl ring, a cyclopentyl ring, or a cyclohexyl ring);

R₂ is chosen from —CH₃, —CH₂CH₃, —CH₂CH₂CH₃, and —CH₂CH₂CH₂CH₃, (or can be taken together with R₁ to give a cyclopropyl ring, a cyclobutyl ring, a cyclopentyl ring, or a cyclohexyl ring);

R₃ is chosen from —COOH, —COOR₆, —CONH₂, —CONHR₆, —CONR₆R₇, —CONHSO₂R₆, tetrazolyl, and a —COOH bioisostere;

R₄ is chosen from —Cl, —F, —Br, —I, —CF₃, —OCF₃, —SCF₃, —OCH₃, —OCH₂CH₃, —CN, —CH═CH₂, —CH₂OH, and —NO₂;

R₅ is chosen from —Cl, —F, —Br, —I, —CF₃, —OCF₃, —SCF₃, —OCH₃, —OCH₂CH₃, —CN, —CH═CH₂, —CH₂OH, and —NO₂;

R₆ is chosen from —CH₃, —CH₂CH₃, —CH₂CH₂CH₃, and —CH₂CH₂CH₂CH₃;

R₇ is chosen from —CH₃, —CH₂CH₃, —CH₂CH₂CH₃, and —CH₂CH₂CH₂CH₃;

m is an integer chosen from 0, 1, 2, and 3; and

n is an integer chosen from 0, 1, 2, and 3.

As used herein, the term “Aβ₄₂ lowering agent” refers to an agent capable of reducing Aβ₄₂ levels according to Example 3 below. The level of Aβ₄₂ can be reduced by a detectable amount. For example, treatment with an Aβ₄₂ lowering agent may lead to at least about 0.5, 1, 3, 5, 7, 15, 20, 40, 50, or more than about 50% reduction in the level of Aβ₄₂ when compared with that in the absence of the Aβ₄₂ lowering agent. Preferably, the Aβ₄₂ lowering agent is capable of producing at least a 20% reduction in the level of Aβ₄₂ generated when compared to that in the absence of Aβ₄₂ lowering agent. More preferably, the Aβ₄₂ lowering agent leads to at least a 40% reduction the level of Aβ₄₂ when compared to that in the absence of an Aβ₄₂ lowering agent.

Examples of Aβ₄₂ lowering compounds for use in the invention include those as shown above (and below), including enantiomers, diastereomers, racemates, and pharmaceutically acceptable salts thereof. The compounds described in this invention disclosure can be made by an ordinary artisan skilled in the art of organic chemistry synthesis.

Exemplary compounds of Formulae I-V include, 2-methyl-2 (2-fluoro-4′-trifluoromethylbiphen-4-yl)propionic acid; 2-methyl-2 (2-fluoro-4′cyclohexyl biphen-4-yl)propionic acid;1-(2-fluoro-4′-trifluoromethylbiphenyl-4-yl)cyclopropanecarboxylic acid; 1-(4′-cyclohexyl-2-fluorobiphenyl-4-yl)cyclopropanecarboxylic acid; 1-(4′-benzyloxy-2-fluorobiphenyl-4-yl)cyclopropanecarboxylic acid; 1-(2-fluoro-4′-isopropyloxybiphenyl-4-yl)cyclopropanecarboxylic acid; 1-(2-fluoro-3′-trifluoromethoxybiphenyl-4-yl)cyclopropanecarboxylic acid; 1-(2-fluoro-4′-trifluoromethoxybiphenyl-4-yl)cyclopropanecarboxylic acid; 1-(2-fluoro-3′-trifluoromethylbiphenyl-4-yl)cyclopropanecarboxylic acid; 1-(4′-cyclopentyl-2-fluorobiphenyl-4-yl)cyclopropanecarboxylic acid; 1-(4′-cycloheptyl-2-fluorobiphenyl-4-yl)cyclopropanecarboxylic acid; 1-(2′-cyclohexyl-2-fluorobiphenyl-4-yl)cyclopropanecarboxylic acid; 1-(2-fluoro-4′-hydroxybiphenyl-4-yl)cyclopropanecarboxylic acid; 1-[2-fluoro-4′-(tetrahydropyran-4-yloxy)biphenyl-4-yl]-cyclopropane-carboxylic acid; 1-(2,3′,4′-trifluorobiphenyl-4-yl)cyclopropanecarboxylic acid; 1-(3′,4′-dichloro-2-fluorobiphenyl-4-yl)cyclopropanecarboxylic acid; 1-(3′,5′-dichloro-2-fluorobiphenyl-4-yl)cyclopropanecarboxylic acid 1-(3′-chloro-2,4′-difluorobiphenyl-4-yl)cyclopropanecarboxylic acid; 1-(4-benzo[b]thiophen-3-yl-3-fluorophenyl)cyclopropanecarboxylic acid; 1-(2-fluoro-4′-prop-2-inyloxy-biphenyl-4-yl)-cyclopropanecarboxylic acid; 1-(4′-cyclohexyloxy-2-fluoro-biphenyl-4-yl)-cyclopropanecarboxylic acid; 1-[2-fluoro-4′-(tetrahydropyran-4-yl)-biphenyl-4-yl]-cyclopropanecarboxylic acid; 1-[2-fluoro-4′-(4-oxo-cyclohexyl)-biphenyl-4-yl]-cyclopropanecarboxylic acid; 2-(2″-fluoro-4-hydroxy-[1,1′:4′,1″]tert-phenyl-4″-yl)-cyclopropanecarboxylic acid; 1-[4′-(4,4-dimethylcyclohexyl)-2-fluoro[1,1′-biphenyl]-4-yl]-cyclopropane-carboxylic acid; 1-[2-fluoro-4′-[[4-(trifluoromethyl)benzoyl]amino][1,1′-biphenyl]-4-yl]-cyclopropanecarboxylic acid; 1-[2-fluoro-4′-[[4-(trifluoromethyl)cyclohexyl]oxy][1,1′-biphenyl]-4-yl]- cyclopropanecarboxylic acid; 1-[2-fluoro-4′-[(3,3,5,5-tetramethylcyclohexyl)oxy][1,1′-biphenyl]-4-yl]-cyclopropanecarboxylic acid; 1-[4′-[(4,4-dimethylcyclohexyl)oxy]-2-fluoro[1,1′-biphenyl]-4-yl]-cyclopropanecarboxylic acid; 1-(2,3′,4″-trifluoro[1,1′:4′,1″-tert-phenyl]-4-yl)-cyclopropanecarboxylic acid; 1-(2,2′,4″-trifluoro[1,1′:4′,1″-tert-phenyl]-4-yl)-cyclopropanecarboxylic acid; 1-(2,3′-difluoro-4″-hydroxy[1,1+:4′,1″-tert-phenyl]-4-yl)-cyclopropane-carboxylic acid; 1- (2,2′-difluoro-4″-hydroxy[1,1′: 4′,1″-tert-phenyl]-4-yl)-cyclopropane- carboxylic acid; 2-(2-fluoro-3′,5′-bis (chloro)biphen-4-yl)propionic acid amide; 2-(2-fluoro-4′-trifluoromethylbiphen-4-yl)propionic acid; 2-(2-fluoro-3′-trifluoromethylbiphen-4-yl)propionic acid; 2-(2-fluoro-3′,5′-bis(trifluoromethyl)biphen-4-yl)propionic acid; 2-(4′-cyclohexyl-2-fluorobiphen-4-yl)propionic acid; 2-(2-Fluoro-1,1′-biphenyl-4-yl)-2-methylpropanoic acid; 2-Methyl-2-(3-phenoxy-phenyl)-propionic acid; 2-(4-Isobutyl-phenyl)-2-methyl-propionic acid; 2-(6-Chloro-9H-carbazol-2-yl)-2-methyl-propionic acid; 2-[1- (4-Chloro-benzoyl)-5-methoxy-2-methyl-1H-indol-3-yl]-2-methyl-propionic acid; and 5-[1-(2-Fluoro-biphenyl-4-yl)-1-methyl-ethyl]-2H-tetrazole.

In specific embodiments, the compound is (R)-2-(2-fluoro-4-biphenyl)propionic acid. As used herein, the term “(R)-2-(2-fluoro-4-biphenyl)propionic acid” refers to the free acid form of (R)-2-(2-fluoro-4-biphenyl)propionic acid and molar equivalents of various salt forms, substantially free of (S)-2-(2-fluoro-4-biphenyl)propionic acid. (R)-2-(2-fluoro-4-biphenyl)propionic acid is the “R” enantiomer of flurbiprofen ((R,S)-2-(2-fluoro-4-biphenyl)propionic acid). (R)-2-(2-fluoro-4-biphenyl)propionic acid can be obtained from resolving racemic flurbiprofen or through enantioselective or enantiospecific syntheses. The R-isomer of flurbiprofen ((R)-2-(2-fluoro-4-biphenyl)propionic acid), or a desired enantiomeric excess of (R)-2-(2-fluoro-4-biphenyl)propionic acid, can then be obtained by resolving the racemic flurbiprofen according to well-known methods, and is also commercially available (e.g., Caymen Chemical, Ann Arbor, Mich.). Methods of resolving (R)-2-(2-fluoro-4-biphenyl)propionic acid from the racemate are disclosed in U.S. Pat. No. 5,599,969 to Hardy et al. which discloses reacting racemic flurbiprofen with α-methylbenzylamine to form an isolatable salt of (R)-2-(2-fluoro-4-biphenyl)propionic acid. U.S. Pat. No. 4,209,638 to Boots Co. discloses a process for resolving 2-arylpropionic acids, which include flurbiprofen, by mixing the racemate with a chiral organic nitrogenous base under certain conditions followed by recovery and separation of the diastereomeric salts. Other patents disclosing processes for resolving racemic arylpropionic acids include U.S. Pat. No. 4,983,765 to PAZ; U.S. Pat. No. 5,015,764 to Ethyl Corp.; U.S. Pat. No. 5,235,100 to Ethyl Corp.; U.S. Pat. No. 5,574,183 to Albemarle Corp.; and U.S. Pat. No. 5,510,519 to Sumitomo Chemical Company.

The compound (R)-2-(2-fluoro-4-biphenyl)propionic acid is substantially free of (S)-2-(2-fluoro-4-biphenyl)propionic acid. For example, (R)-2-(2-fluoro-4-biphenyl)propionic acid may be at least about 90%, at least about 95%, at least about 99%, or at least about 99.9% by weight of the total 2-(2-fluoro-4-biphenyl)propionic acid (S+R) administered to a patient according to the invention.

The term “bioisostere”, as used herein, generally refers to compounds or moieties that have chemical and physical properties producing broadly similar biological properties. For example, —COOH bioisosteres include, but are not limited to, a carboxylic acid ester, amide, tetrazole, oxadiazole, isoxazole, hydroxythiadiazole, thiazolidinedione, oxazolidinedione, sulfonamide, sulfonylcarboxamide, phosphonic acid, phosphonamide, phosphinic acid, sulfonic acid, acyl sulfonamide, mercaptoazole, and cyanamide.

In specific embodiments, overactive bladder is treated. As used herein, overactive bladder refers to a chronic condition resulting from overactivity of the detrusor muscles, wherein the bladder initiates contraction too early while filling with urine. Overactive bladder can be neurogenic or non-neurogenic. Neurogenic overactive bladder is a type of overactive bladder which occurs as a result of detrusor muscle overactivity referred to as detrusor hyperreflexia, secondary to neurological disorders. Non-neurogenic overactive bladder occurs as a result of detrusor muscle overactivity referred to as detrusor muscle instability, which can be idiopathic or may arise from non-neurological abnormalities such as bladder stones, muscle disease, urinary tract infection, or drug side effect.

In other embodiments urinary incontinence is treated. As used herein, urinary incontinence refers to the inability to control the passage of urine.

In additional embodiments, symptoms of overactive bladder and urinary incontinence selected form urinary frequency, urinary urgency, nocturia, and enuresis are treated. As used herein, urinary frequency refers to urinating more frequently than the patient desires. Because the frequency of desired urination varies substantially with each patient, a patient's desired frequency may be further defined as the median number of times the patient urinated per day during a normal or desirable time period. Urinary urgency, as used herein, refers to sudden strong urges to urinate with little or no chance to postpone the urination. As used herein, nocturia refers to being awakened from sleep to urinate more frequently than the patient desires. Enuresis, as used herein, refers to involuntary discharge of urine which can be complete or incomplete.

In another aspect of the invention, compositions and methods are provided for treating or slowing the onset of overactive bladder and/or urinary incontinence, or symptoms thereof, comprising identifying and administering to a subject with Alzheimer's disease who is in need of such treatment a therapeutically effective amount of an Aβ₄₂ lowering agent, such as a compound according to Formulae I-V.

Individuals with Alzheimer's disease (AD) can be diagnosed by any method available to the ordinary artisan skilled is such diagnoses. For example, progression or severity of AD can be determined using the Mini-Mental State Exam (MMSE; see Mohs et al. Int. Psychogeriatr. 8:195-203 (1996)); ADAS-Cog (Alzheimer Disease Assessment Scale-Cognitive; see Galasko et al. Alzheimer Dis Assoc Disord, 11 suppl 2:S33-9 (1997)); Behavioral Pathology in Alzheimer's Disease Rating Scale (BEHAVE-AD); Blessed Test; CANTAB—Cambridge Neuropsychological Test Automated Battery; CERAD (The Consortium to Establish a Registry for Alzheimer's Disease) Clinical and Neuropsychological Tests (includes MMSE); Clock Draw Test; Cornell Scale for Depression in Dementia (CSDD); Geriatric Depression Scale (GDS); Neuropsychiatric Inventory (NPI); the 7 Minute Screen; the Alzheimer's Disease Cooperative Study Activities of Daily Living scale (ADCS-ADL; see McKhann et al. Neurology 34:939-944 (1984)); the DSM-IV (Diagnostic and Statistical Manual of Mental Disorders—Fourth Edition (DSM-IV), published by the American Psychiatric Association, Washington D.C., 1994); or the NINCDS-ADRDA criteria (see Folstein et al. J. Psychiatr. Res. 12:189-198 (1975)).

Individuals who may particularly benefit from the compositions and methods of the invention include those individuals diagnosed as having mild to moderate Alzheimer's disease according to a medically-accepted diagnosis, such as, for example the NINCDS-ADRDA criteria. Individuals diagnosed as having probable AD can be identified as having a mild-to-moderate form of the disease by an accepted measure of cognitive function such as the MMSE. In addition, methods that allow for evaluating different regions of the brain and estimating plaque and tangle frequencies can be used. These methods are described by Braak et al. Acta Neuropathol 82:239-259 (1991); Khachaturian Arch. Neuro. 42:1097-1105 (1985); Mirra et al. (1991) Neurology 41:479-486; and Mirra et al. Arch Pathol Lab Med 117:132-144 (1993). The severity of AD is generally determined by one of the initial tests provided above. For example, MMSE scores of 26-19 indicate mild AD, while scores from 18-10 indicate moderate AD.

Diagnoses of Alzheimer's disease based on these tests are recorded as presumptive or probable, and may optionally be supported by one or more additional criteria. For example, a diagnosis of Alzheimer's disease may be supported by evidence of a family history of AD; non-specific changes in EEG, such as increased slow-wave activity; evidence of cerebral atrophy on CT with progression documented by serial observation; associated symptoms such as depression, insomnia, incontinence, delusions, illusions, hallucinations, catastrophic verbal, emotional or physical outbursts, sexual disorders, weight loss, and/or attendant neurologic abnormalities, such as increased muscle tone, myoclonus or gait disorder, etc.

Additionally, amyloid deposits, generally associated with AD, may be detected through the use of positron emission tomography (PET) using an amyloid-specific tracer such as Pittsburgh Compound-B (PIB). See Klunk et al., Ann. Neurol. 55(3):306-309 (2004). Increased amyloid deposits in the frontal, parietal, temporal and occipital cortices, and in the striatum, relative to normal brain tissue, as visualized, for example by PIB, support a diagnosis of AD. Generally, a greater number and density of amyloid deposits indicates more advanced AD.

Thus, in certain embodiments, an Aβ₄₂ lowering agent is administered to an individual diagnosed as having mild to moderate Alzheimer's disease to treat or slow the onset of overactive bladder and/or urinary incontinence, or symptoms thereof. In a more specific embodiment, said individual is diagnosed by a cognitive test as having mild to moderate AD. In a more specific embodiment, said cognitive test is the Mini-Mental State Exam (MMSE). In an even more specific embodiment, said individual has a score in said MMSE of from 26 to 19, inclusive. In another more specific embodiment, said individual has a score in said MMSE of from 18 to 10, inclusive. In another specific embodiment, said individual has a score in said MMSE of 26 to 10, inclusive.

In yet another embodiment, an effective amount of an Aβ₄₂ lowering agent is administered to an individual having or suspected of having mild cognitive impairment (MCI) to treat or slow the onset of overactive bladder and/or urinary incontinence, or symptoms thereof. Mild cognitive impairment is a clinical condition between normal aging and Alzheimer's disease characterized by memory loss greater than expected for the particular age of the individual yet the individual does not meet the currently accepted definition for probable Alzheimer's disease. See, e.g., Petersen et al. Arch. Neurol. 58:1985-1992 (2001); Petersen Nature Rev. 2:646-653 (2003); and Morris et al. J. Mol. Neuro. 17:101-118 (2001). Typically, patients having MCI first complain of or have a loss of memory. Preferably an individual associated with the patient can corroborate the memory deficit. Furthermore, general cognition is not sufficiently impaired to cause concern about more widespread cognitive disorder and although daily living activities may be affected that are not significantly impaired and the patients are not demented.

In specific embodiments, the Aβ₄₂ lowering agent, is (R)-2-(2-fluoro-4-biphenyl)propionic acid. In certain embodiments, overactive bladder is treated. In other embodiments urinary incontinence is treated. In additional embodiments, symptoms of overactive bladder and urinary incontinence selected form urinary frequency, urinary urgency, nocturia, and enuresis are treated.

In another aspect of the invention, compositions and methods are provided for treating overactive bladder or urinary incontinence, or symptoms thereof, comprising identifying and administering to a subject in need a therapeutically effective amount of a compound according to Formulae I-V in combination with at least one additional therapeutic agent. In certain embodiments, the subject in need has Alzheimer's disease.

Additional therapeutic agents suitable for use in the methods and pharmaceutical compositions described herein include, but are not limited to, an antimuscarinic agents such as oxybutynin, DITROPAN®, tolterodine, flavoxate, propiverine, or trospium; a muscosal surface protectant such as ELMIRON®; an antihistamine such as hydroxyzine hydrochloride or pamoate; an anticonvulsant such as NEURONT® or KLONOPIN®; a muscle relaxant such as VALIUM®; a bladder antispasmodic such as URIMAX®; a tricyclic antidepressant such as imipramine; a nitric oxide donor such as nitroprusside, a β₃-adrenergic receptor agonist; a bradykinin receptor antagonist; a neurokinin receptor antagonist; a sodium channel modulator such as a TTX-R sodium channel modulator, an activity dependent sodium channel modulator, or a Cav2.2 subunit calcium channel modulator.

Additional therapeutic agents used in combination with compounds of Formulae I-V may include the specific agents disclosed herein as well as pharmaceutically acceptable acids, salts, esters, amides, prodrugs, active metabolites, or other derivatives thereof. Generally, the additional therapeutic agent will be one that is useful for treating the disorder of interest. Preferably, the additional therapeutic agent does not diminish the effects of the primary agent(s) and/or potentiates the effect of the primary agent(s).

Use of one or more additional therapeutic agents in combination with a compound of Formulae I-V can result in less of any of the Formulae I-V compounds and/or less of the additional agent being needed to achieve therapeutic efficacy. In some instances, use of less of an agent can be advantageous in that it provides a reduction in undesirable side effects.

As used herein, “antimuscarinic agent” means any muscarinic acetylcholine receptor antagonist. Exemplary antimuscarinic agents include:
(a) Oxybutynin (Ditropan®, Ditropan XL® (extended-release formula));
(b) Oxybutynin metabolites and isomers such as N-desethyl-oxybutynin and S-oxybutynin (see e.g. U.S. Pat. Nos. 5,736,577 and 5,532,278);
(c) Tolterodine (Detrol®, Detrol LA® (time-released));
(d) Hyoscyamine (Cystospaz®, Cystospaz-M® (time-released), Levsin®, Levbid® (sublingual), Levsinex® (time-released));
(e) Flavoxate (Urispas®);
(f) Dicyclomine (Bentyl®);
(g) Propantheline (Pro-Banthine®);
(h) Solifenacin, Solifenacin succinate, and Solifenacin monohydrochloride;
(i) Propiverine (Detrunorm®);
(j) Trospium chloride;
(j) Darifenacin (Daryon®);
(k) d, 1 (racemic) 4-diethylamino-2-butynyl phenylcyclohexylglycolate;
(l) (R)-N,N-diisopropyl-3-(2-hydroxy-5-methylphenyl)-3-phenylpropanamine L-hydrogen tartrate;
(m) (+)-(1S,3′R)-quinuclidin-3′-yl-1-phenyl-1,2,3,4-tetrahydro-isoquinoline-2-carboxylate monosuccinate;
(n) Alpha (+)-4-(Dimethylamino)-3-methyl-1,2-diphenyl-2-butanol proprionate;
(o) 1-methyl-4-piperidyl diphenylpropoxyacetate;
(p) 3-hydroxyspiro[1H,5H-nortropane-8,1′-pyrrolidinium benzilate;
(q) 4 amino-piperidine containing compounds as disclosed in Diouf et al. (2002) Bioorg. Med. Chem. Lett. 12: 2535-9;
(r) Pirenzipine;
(s) Methoctramine;
(t) 4-diphenylacetoxy-N-methyl piperidine methiodide;
(u) Tropicamide;
(v) (2R)-N-[1-(6-aminopyridin-2-yhnethyl)piperidin-4-yl]-2-[(1R)-3,3-difluorocyclopentyl]-2-hydroxy-2-phenylacetamide;
(w) PNU-200577 ((R)-N,N-diisopropyl-3-(2-hydroxy-5-hydroxyrmethylphenyl)-3-phenylpropanamine);
(x) KRP-197 (4-(2-methylimidazolyl)-2,2-diphenylbut amide);
(y) Fesoterodine; and
(z) SPM 7605 (the active metabolite of Fesoterodine).

The identification of further compounds that have antimuscarinic activity and would therefore be useful in the present invention can be determined by performing muscarinic receptor binding specificity studies as described by Nilvebrant (2002) Pharmacol Toxicol. 90: 260-267 or cystometry studies as described by Modiri et al. (2002) Urology 59: 963-8.

The term “β₃-adrenergic receptor agonist” is used in its conventional sense to refer to a compound that binds to and agonizes β₃ adrenergic receptors. Compounds that have been identified as β₃ adrenergic agonist agents and are useful in the present invention include, but are not limited to:
a. TT-138 and phenylethanolamine compounds as disclosed in U.S. Pat. No. 6,069,176, PCT Publication No. WO 97/15549 and available from Mitsubishi Phanna Corp.;
b. FR-149174 and propanolamine derivatives as disclosed in U.S. Pat. Nos. 6,495,546 and 6,391, 915 and available from Fujisawa Pharmaceutical Co.;
c. KUC-7483, available from Kissei Pharmaceutical Co.,;
d. 4′-hydroxynorephedrine derivatives such as 2-2-chloro-4-(2-((1S,2R)-2-hydroxy-2-(4-hydroxyphenyl)-1-methylethylalnino) ethyl)-phenoxy acetic acid as disclosed in Tanaka et al. (2003) J. Med. Chem. 46: 105-12;
e. 2-amino-1-phenylethanol compounds, such as BRL35135 ((R*R*)-(.±.)-[4-[2-[2-(3-chlorophenyl)-2-ydroxyethylamino]propyl]phenoxy]acetic acid methyl ester hydrobromide salt as disclosed in Japanese Patent Publication No. 26744 of 1988 and European Patent Publication No. 23385), and SR58611A ((RS)-N-(7-ethoxycarbonylmethoxy-1,2,3,4-tetrahydronaphth-2-yl)-2-(3-chlorophenyl)-2-hydroxyethanamine hydrochloride as disclosed in Japanese Laid-open Patent Publication No. 66152 of 1989 and European Laid-open Patent Publication No. 255415);
f. GS 332 (Sodium (2R)-[3-[3-[2-(3 Chlorophenyl)-2-hydroxyethylamino]cyclohexyl]phenoxy]acetate) as disclosed in Iizuka et al. (1998) J. Smooth Muscle Res. 34: 139-49;
g. BRL-37,344 (4-[-[(2-hydroxy-(3-chlorophenyl)ethyl)-amino]propyl]phenoxyacetate) as disclosed in Tsujii et al. (1998) Physio. Behav. 63: 723-728 and available from GlaxoSmithKline;
h. BRL-26830A as disclosed in Takahashi et al. (1992) Jpn Circ. A. 56: 936-942 and available from GlaxoSmithKline;
i. CGP 12177 (4-[3-t-butylamino-2-hydroxypropoxy]benzimidazol-2-one) (a ½ adrenergic antagonist reported to act as an agonist for the 3 adrenergic receptor) as described in Tavernier et al. (1992) J. Pharmacol. Exp. Tlzer. 263: 1083-90 and available from Ciba-Geigy;
j. CL 316243 (R, R-5-[2-[[2-(3-chlorophenyl)-2-hydroxyethyl]amino]propyls-1,3-benzodioxole-2,2-dicarboxylate) as disclosed in Berlan et al. (1994) J. Pharmacol. Exp. Tuer. 268: 1444-51;
k. Compounds having 3 adrenergic agonist activity as disclosed in US Patent Application 20030018061;
l. ICI 215,001 HCl ((S)-4-[2-Hydroxy-3-phenoxypropyl-aminoethoxy]phenoxyacetic acid hydrochloride) as disclosed in Howe (1993) Drugs Future 18: 529 and available from AstraZeneca/ICI Labs;
m. ZD 7114 HCl (ICI D7114; (S)-4-[2-Hydroxy-3-phenoxypropyl-aminoethoxy]-N-(2-methoxyethyl)phenoxyacetamide HCl) as disclosed in Howe (1993) Drugs Future 18: 529 and available from AstraZeneca/ICI Labs;
n. Pindolol (1-(1H-Indol-4-yloxy)-3-[(1-methylethyl)amino]-2-propanol) as disclosed in Blin et al (1994) Mol. Pharmacol. 44: 1094;
o. (S)-(−)-Pindolol ((S)-1-(1H-indol-4-yloxy)-3-[(1-methylethyl)amino]-2-propanol) as disclosed in Walter et al (1984) Naunyn-Schmied. Arch. Pharmacol. 327: 159 and Kallanan (19S9) Eur. J. Pharmacol. 173: 121;
p. SR 59230A HCl (1-(2-Ethylphenoxy)-3-[[(1S)-1,2,3,4-tetrahydro-1-naphthalenyl]amino]-(2S)-2-propanol hydrochloride) as disclosed in Manara et al. (1995) Pharmacol. Comm. 6: 253 and Manara et al. (1996) Br. J. Phannacol. 117: 435 and available from Sanofi-Midy;
q. SR 58611 (N[2s) 7-carb-ethoxymethoxy-1,2,3,4-tetra-hydronaphth]-(2r)-2-hydroxy-2 (3-chlorophenyl)ethamine hydrochloride) as disclosed in Gauthier et al. (1999) J. Pharmacol. Exp. Ther. 290: 687-693 and available from Sanofi Research; and
r. YM178 available from Yamanouchi Pharmaceutical Co.

The identification of further compounds that have β₃ adrenergic agonist activity and would therefore be useful in the present invention can be determined by performing radioligand binding assays and/or contractility studies as described by Zilberfarb et al. (1997) J Cell Sci. 110: 801-807 ; Takeda et al. (1999) J Pharmacol. Exp. Ther. 288: 1367-1373; and Gauthier et al. (1999) J Pharmacol. Exp. Ther. 290: 687-693.

Further, agents for use as additional therapeutic agents include sodium channel modulators, such as TTX-R sodium channel modulators and/or activity dependent sodium channel modulators. TTX-R sodium channel modulators for use in the present invention include but are not limited to compounds that modulate or interact with Nav1. 8 and/or Nav 1. 9 channels.

Sodium channel modulators suitable for use as in the practice of the invention include, but are not limited to propionamides such as Ralfinamide (NW-1029) (as disclosed in U.S. Pat. No. 5,236,957 and U.S. Pat. No. 5,391,577), which is also known as (+)-2 (S)-[4-(2-Fluorobenzyloxy)benzylamino]propionamide and safinamide (as disclosed in U.S. Pat. No. 5,236,957 and U.S. Pat. No. 5,391,577), which is also known as 2 (S)-[4-(3-Fluorobenzyloxy)benzylamino]propionamide methanesulfonate.

Further sodium channel modulators include for example, N-phenylalkyl substituted a-amino carboxamide derivatives in addition to Ralfinamide and Salfinamide as disclosed in U.S. Pat. No. 5,236,957; other N-phenylalkyl substituted a-amino carboxamide derivatives in addition to Ralfinamide and Salfinamide as disclosed in U.S. Pat. No. 5,391,577; substituted 2-benzylamino-2-phenyl-acetamide compounds as disclosed in U.S. Pat. No. 6,303, 819; aryldiazines and aryltriazines such as: sipatrigine (BW-619C; as disclosed in U.S. Pat. No. 5,684,005), which is also known as 4-Amino-2-(4-methylpiperazin-1-yl)-5-(2,3,5-trichlorophenyl)pyrimidine; 2-(4-Methylpiperazin-1-yl)-5-(2,3,5-trichlorophenyl)pyrimidine-4-amine; lamotrigine (as disclosed in U.S. Pat. No. 4,602,017), which is also known as 6-(2,3-Dichlorophenyl)-1,2,4-triazine-3,5-diamine; GW-273293 (as disclosed in U.S. Pat. No. 6,599,905), which is also known as 3-(2,3,5-Trichlorophenyl)pyrazine-2,6-diamine; 4030W92 (as disclosed in U.S. Pat. No. 6,124,308), which is also known as 5-(2,3-Dichlorophenyl)-6-(fluoromethyl)pyrimidine-2,4-diamine; Carbamazepine (as disclosed in U.S. Pat. No. 2,948,718), which is also known as 5H-Dibenz[d, flazepine-5-carboxamide; Oxcarbazepine (as disclosed in U.S. Pat. No. 3,642,775), which is also known as 10-Oxo-10, 11-dihydro-5H-dibenz[b,f]azepine-5-carboxamide; licarbazepine (as disclosed in DE 2011045), which is also known as (±)-10-Hydroxy-10,11 -dihydro-5H-dibenz[b, f]azepine-5-carboxamide; BIA-2-093 (as disclosed in U.S. Pat. No. 5,753,646), which is also known as Acetic acid 5-carbamoyl-10,11-dihydro-5H-d-benzo[b, f]azepin-10(S)-yl ester; ADCI (as disclosed in U.S. Pat. No. 5,196,415), which is also known as (±)-5,10-Imino-10,11-dihydro-5H-dibenzo[a,d]cycloheptene-5-carboxamide; Phenytoin sodium (as disclosed in U.S. Pat. No. 2,409,754) and OROS®-Phenytein (as disclosed in U.S. Pat. No. 4,260,769), which are also known as 5,5-Diphenylhydantoin sodium salt and 5, 5-Diphenyl-2,4-imidazolidinedione salt; Fosphenytoin sodium (as disclosed in U.S. Pat. No. 4,260,769) and phosphenytoin sodium, which are also known as 3-(Hydroxymethyl)-5,5-diphenylhydantoin phosphate ester disodium salt and 5,5-Diphenyl-3-[(phosphonooxy)methyl]-2,4-imidazolidinedione disodium salt; Pilsicainide hydrochloride and analogs thereof (as disclosed in U.S. Pat. No. 4,564,624), which is also known as N-(2,6-Dimethylphenyl)-8-pyrrolizidineacetamide hydrochloride; N-(2,6-Dimetliylphenyl)-1-azabicyclo[3.3.0]octane-5-acetamide hydrochloride; Tocainide (as disclosed in DE 2235745), which is also known as 2-Amino-N-(2,6-dimethylphenyl)propanamide hydrochloride; Flecainide (as disclosed in U.S. Pat. No. 3,900,481), which is also known as N-(2-Piperidylmethyl)-2,5-bis(2,2,2-trifluoroethoxy)benzamide monoacetate; mexiletine hydrochloride (as disclosed in U.S. Pat. No. 3,954,872), which is also known as 1-(2,6-Dimethylphenoxy)-2-propanamine hydrochloride; Ropivacaine hydrochloride (as disclosed in PCT Publication No. WO 85/00599), which is also known as (−)-(S)-N-(n-Propyl)piperidine-2-carboxylic acid 2,6-xylidide hydrochloride monohydrate; (−)-(S)-N-(2,6-Dimethylphenyl)-1-propylpiperidine-2-carboxamide hydrochloride monohydrate; (−)-(S)-1-Propyl-2′,6′-pipecoloxylidide hydrochloride monohydrate; Lidocaine (as disclosed in U.S. Pat. No. 2,441,498), which is also known as 2-(diethylamino)-N-(2,6-dimethylphenyl) acetamide; mepivacaine (as disclosed in U.S. Pat. No. 2,799,679), which is also known as N-(2,6-dimethylphenyl)-1-methyl-2-piperidinecarboxamide; bupivacaine (as disclosed in U.S. Pat. No. 2,955,111), which is also known as 1-butyl-N-(2,6-dimethylphenyl)-2-piperidinecarboxamide; Prilocaine (as disclosed in U.S. Pat. No. 3,160,662), also known as N-(2-methylphenyl)-2-(propylamino)propanamide; etidocaine (as disclosed in U.S. Pat. No. 3,812,147), which is also known as N-(2,6-dimethylphenyl)-1-methyl-2-piperidinecarboxamide, tetracaine (as disclosed in U.S. Pat. No. 1,889,645), which is also known as 4-(butylamino)benzoic acid 2-(diethylmino)ethyl ester; dibucaine (as disclosed in U.S. Pat. No. 1,825,623), which is also known as 2-butoxy-N-[2-(diethylamino)-ethyl]-4-quinolinecarboxamide; Soretolide, which is also known as 2,6-Dimethyl-N-(5-metllylisozaxol-3-yl)benzamide ; RS-132943 (as disclosed in U.S. Pat. No. 6,110,937), which is also known as 3 (S)-(4-Bromo-2,6- dimethylphenoxymethyl)-1-methylpiperidine hydrochloride The identification of other agents that have affinity for TTX-R sodium channels or proteins associated with TTX-R sodium channels and would be useful in the present invention can be determined by methods that measure functional TTX-R channel activity such as sodium flux as disclosed in Stallcup, W B (1979) J. Physio. 286: 525-40 or electrophysiological approaches as disclosed in Weiser and Wilson (2002) Mol. Pharmacol. 62: 433-438. The identification of other agents that exhibit activity-dependent modulation of sodium channels and would be useful in the present invention can be determined by methods as disclosed in Li et al., (1999) Molecular Pharmacology 55: 134-141.

Further, agents for use as additional therapeutic agents include “Cav2.2 subunit calcium channel modulators” which are capable of binding to the Cav2.2 subunit of a calcium channel to produce a physiological effect, such as opening, closing, blocking, up-regulating expression, or down-regulating expression of the channel. Unless otherwise indicated, the term “Cav2.2 subunit calcium channel modulator” is intended to include amino acid compounds, peptide, nonpeptide, peptidomimetic, small molecular weight organic compounds, and other compounds that modulate or interact with the Cav2.2 subunit of a calcium channel (e. g., a binding event) or proteins associated with the Cav2.2 subunit of a calcium channel (e. g., a binding event) such as anchor proteins.

Cav2. 2 subunit calcium channel modulators useful as an additional therapeutic agent in the practice of the invention include, but are not limited to:
a. ω-conotoxin GVIA, ω-conotoxin MVIIA, ω-conotoxin CNVDA, ω-conotoxin CVIID, and ω-conotoxin AM336;
b. Cilnidipine;
C. Amlodipine;
d. L-cystine derivative 2A;
e. o-agatoxin IVA;
j. N,N-dialkyl-dipeptidylamines;
k. Levetiracetam;
l. Ziconotide (SNX-111);
m. (S)-alpha-ethyl-2-oxo-1-pyrrolidineacetamide (disclosed in U.S. Pat. Nos. 4,943,639, 4,837,223, and 4,696,943);
n. Substituted peptidylamines as disclosed in PCT Publication No. WO 98/54123;
o. PD-173212;
p. Reduced dipeptide analogues as disclosed in U.S. Pat. No. 6,316,440 and PCT Publication No. WO 00/06559;
q. Amino acid derivatives as disclosed in PCT Publication No. WO 99/02146;
r. Benzazepine derivatives as disclosed in Japanese Publication No. JP 2002363163;
s. Compounds disclosed in PCT Publication No. WO 02/36567;
t. Compounds disclosed in PCT Publication No. WO 03/018561;
u. Compounds disclosed in U.S. Patent Publication No. 2004009991 and PCT Publication No. WO 02/22588;
v. Dihydropyridine derivatives as disclosed in U.S. Pat. No. 6,610,717, U.S. Patent Publication No. 2002193605, and PCT Publication No. WO 00/78720;
w. Diarylalkene and diarylalkane derivatives as disclosed in PCT Publication No. WO 03/018538.

Additional Cav2.2 subunit calcium channel modulators useful as an additional therapeutic agent in the practice of the invention include, but are not limited to non-peptide, and peptidomimetic drug-like molecules that bind to Cav2.2-containing calcium channels as disclosed in Lewis et al. (2000) J. Biol. Chem. 10: 35335-44; Smith et al. (2002) Pain 96: 119-27; Takahara et al. (2002) Eur. J. Pharmacol. 434: 43-7; Favreau et al. (2001) Biochemistry, 40: 14567-575; Seko et al. (2001) Bioorg Med. Chem. Lett. 11: 2067-70; Hu et al. (2000) Bioorg. Med. Chem. Lett. 8: 1203-12; Lew et al. (1997) J. Biol. Chem. 272: 12014-23.

The identification of other agents that have affinity for the Cav2.2 subunit of a calcium channel and would be useful in the present invention can be determined by performing Cav2.2 subunit binding affinity, electrophysiolgic, and/or other screening methods as described in Feng et al. (J Biol. Chem., 278: 20171-20178, 2003), Feng et al. (J. Biol. Chem., 276: 15728-15735, 2001), Favreau et al. (Biochemistry, 40: 14567-575, 2001), and/or U.S. Pat. No. 6,387,897 assigned to NeuroMed Technologies Inc.

The term “spasmolytic” (also known as “antispasmodic”) is used in its conventional sense to refer to a compound that relieves or prevents muscle spasms, especially of smooth muscle. In general, spasmolytics have been implicated as having efficacy in the treatment of bladder disorders (See, e. g., Talceda et al. (2000) A. Pharmacol. Exp. Ther. 293: 939-45).

Compounds that have been identified as spasmolytic agents and are useful in the present invention include, but are not limited to:
a. a-a-diphenylacetic acid-4-(N-methyl-piperidyl)esters as disclosed in U.S. Pat. No. 5,897,875;
b. Human and porcine spasmolytic polypeptides in glycosylated form and variants thereof as disclosed in U.S. Pat. No. 5,783,416;
c. Dioxazocine derivatives as disclosed in U.S. Pat. No. 4,965,259;
d. Quaternary 6,11-dihydro-dibenzo-[b,e]-thiepine-11-N-alkylnorscopine ethers as disclosed in U.S. Pat. No. 4,608,377;
e. Quaternary salts of dibenzo[1,4]diazepinones, pyrido-[1,4]benzodiazepinones, pyrido[1,5]benzodiazepinones as disclosed in U.S. Pat. No. 4,594,190;
f. Endo-8,8-dialkyl-8-azoniabicyclo (3.2.1) octane-6,7-exo-epoxy-3-alkyl-carboxylate salts as disclosed in U.S. Pat. No. 4,558,054;
g. Pancreatic spasmolytic polypeptides as disclosed in U.S. Pat. No. 4,370,317;
h. Triazinones as disclosed in U.S. Pat. No. 4,203,983;
i. 2-(4-Biphenylyl)-N-(2-diethylaminoalkyl)propionamide as disclosed in U.S. Pat. No. 4,185,124;
k. Aralkylamino carboxylic acids as disclosed in U.S. Pat. No. 4,163,060;
l. Aralkylamino sulfones as disclosed in U.S. Pat. No. 4,034,103;
m. Smooth muscle spasmolytic agents as disclosed in U.S. Pat. No. 6,207,852; and
n. Papaverine.

The identification of further compounds that have spasmolytic activity and would therefore be useful in the present invention can be determined by performing bladder strip contractility studies as described in U.S. Pat. No. 6,207,852; Noronha-Blob et al. (1991) J. Pharmacol. Exp. Ther. 256: 562-567, and/or Kachur et al. (1988) J. Pharmacol. Exp. Ther. 247: 867-872.

The term “neurokinin receptor antagonist”0 is used in its conventional sense to refer to a compound that binds to and antagonizes neurokinin receptors. Suitable neurokinin receptor antagonists for use in the present invention that act on the NK1 receptor include, but are not limited to: 1-imino-2-(2-methoxy-phenyl)-ethyl)-7,7-diphenyl-4-perhydroisoindolone (3aR,7aR) (“RP 67580“); 2S,3S-cis-3-(2-methoxybenzylamino)-2-benzhydrylquinuclidine (“CP 96,345”); and (aR,9R)-7-[3,5-bis(trifluoromethyl)benzyl]-8,9,10,11-tetrahydro-9-methyl-5-(“RP 67580”); 2S, 3S-cis-3-(2-methoxybenzylamino)-2-benzhydrylquinuclidine (“CP 96, 345”); and (aR, 9R)-7-[3,5-bis (trifluoromethyl)benzyl]-8,9,10,11-tetrahydro-9-methyl-5-(4-methylphenyl)-7H-[1,4]diazocino[2,1-g][1,7]naphthyridine-6,13-dione) (“TAK-637”).

Suitable neurokinin receptor antagonists for use in the present invention that act on the NK2 receptor include but are not limited to: ((S)-N-methyl-N-4-4-acetylamino-4-phenylpiperidino)-2-(3,4-dichlorophenyl)butylbenzamide (“SR 48968”); Met-Asp-Trp-Phe-Dap-Leu (“MEN 10,627”); and cyc(Gln-Trp-Phe-Gly-Leu-Met) (“L 659,877”). The identification of further compounds that have neurokinin receptor antagonist activity and would therefore be useful in the present invention can be determined by performing binding assay studies as described in Hopkins et al. (1991) Biochem. Biophys. Res. Comm. 180: 1110-1117; and Aharonyetal et al. (1994) Mol. Pharmacol. 45: 9-19.

The term “bradykinin receptor antagonist” is used in its conventional sense to refer to a compound that binds to and antagonizes bradykinin receptors. Suitable bradykinin receptor antagonists for use in the present invention that act on the B1 receptor include but are not limited to: des-arglOHOE 140 (available from Hoechst Pharmaceuticals) and des-Arg9bradykinin (DABK). Suitable bradykinin receptor antagonists for use in the present invention that act on the B2 receptor include but are not limited to: D-Phe7-BK; D-Arg-(Hyp3-Thi5, 8-D-Phe7)-BK (“NPC 349”); D-Arg-(Hyp3-D-Phe7)-BK (“NPC 567”); D-Arg-(Hyp3-Thi5-D-Tic7-Oic8)-BK (“HOE 140”); H-DArg-Arg-Pro-Hyp-Gly-Thi-c (Dab-DTic-Oic-Arg)c(7gamma-10alpha) (“MEN11270”); H-DArg-Arg-Pro-Hyp-Gly-Thi-Ser-DTic-Oic-Arg-OH (“Icatibant”); (E)-3-(6-acetamido-3-pyridyl)-N-[N-[2,4-dichloro-3-[(2-methyl-8-quinolinyl)oxymethyl]phenyl]-N-methylaminocarbonyhnethyl]acrylamide (“FR173567”); and WIN 64338. These compounds are more fully described in Perkins, M. N., et. al., Pain, supra; Dray, A., et. al., Treads Neurosci., supra; and Meini et al. (2000) Eur. J Pharmacol. 388: 177-82. The identification of further compounds that have bradykinin receptor antagonist activity and would therefore be useful in the present invention can be determined by performing binding assay studies as described in Manning et al. (1986) J. Pharmacol. Exp. Ther. 237: 504 and U.S. Pat. No. 5,686,565.

The term “nitric oxide donor” is used in its conventional sense to refer to a compound that releases free nitric oxide when administered to a patient. Suitable nitric oxide donors for the practice of the present invention include but are not limited to:
a. Nitroglycerin;
b. Sodium nitroprusside;
c. FK 409 (NOR-3);
d. FR 144420 (NOR-4);
e. 3-morpholinosydnonimine;
f. Linsidomine chlorohydrate (“SIN-1”);
g. S-nitroso-N-acetylpenicillamine (“SNAP”);
h. AZD3582 (CINOD lead compound), NCX 4016, NCX 701, NCX 1022, HCT 1026, NCX 1015, NCX 950, NCX 1000, NCX 1020, AZD 4717, NCX 1510/NCX 1512, NCX 2216, and NCX 4040 (all available from NicOx S.A.); and
i. Nitric oxide donors as disclosed in U.S. Pat. Nos. 5,155,137, 5,366,997, 5,405,919, 5,650,442, 5,700,830, 5,632,981, 6,290,981, 5,691,423 5,721,365, 5,714,511, 6,511,911, and 5,814,666.

The identification of further compounds that have nitric oxide donor activity and would therefore be useful in the present invention can be determined by release profile and/or induced vasospasm studies as described in U.S. Pat. Nos. 6,451,337 and 6,358,536, Moon (2002) IBJU Int. 89: 942-9 and Fathian-Sabet et al. (2001) J. Urol. 165: 1724-9.

In specific embodiments, the compound to be administered in combination with an additional therapeutic agent is is (R)-2-(2-fluoro-4-biphenyl)propionic acid.

In certain embodiments, overactive bladder is treated. In other embodiments urinary incontinence is treated. In additional embodiments, symptoms of overactive bladder and urinary incontinence selected form urinary frequency, urinary urgency, nocturia, and enuresis are treated.

The compounds of Formulae I-V and the overactive bladder or urinary incontinence therapeutic agents may be administered separately or together in a single composition. The compounds of Formulae I-V and the overactive bladder or urinary incontinence therapeutic agents may be administered at the same time or may be administered at different times of the day.

The compounds for use in the methods of the invention and compositions of the invention include all compositions wherein the compounds of the present invention are contained in an amount that is effective to achieve its intended purpose. While individual needs vary, determination of optimal ranges of effective amounts of each component is within the skill of the art. Typically, the compounds may be administered to animals, e.g., mammals, orally at a dose of 0.0025 to 50 mg/kg of body weight, per day, or an equivalent amount of the pharmaceutically acceptable salt thereof, to a mammal being treated. In one example, approximately 0.01 to approximately 10 mg/kg of body weight is orally administered. For intramuscular injection, the dose is generally approximately one-half of the oral dose. For example, a suitable intramuscular dose would be approximately 0.0025 to approximately 25 mg/kg of body weight, and from approximately 0.01 to approximately 5 mg/kg of body weight. If an overactive bladder or urinary incontinence therapeutic agent is also administered, it is administered in an amount that is effective to achieve its intended purpose. The amounts of such overactive bladder or urinary incontinence therapeutic agents effective for treating such disorders are well known to those skilled in the art.

Exemplary daily dosages of compounds of Formulae I-V, such as (R)-2-(2-fluoro-4-biphenyl)propionic acid, are from about 1 mg to about 2000 mg, from about 1 mg to about 1600 mg, from about 1 mg to about 800 mg, and from 1 mg to about 600 mg. Additional exemplary daily dosages of compounds of Formulae I-V, such as (R)-2-(2-fluoro-4-biphenyl)propionic acid, are at least 1600 mg/day, at least 800 mg/day, at least 600 mg/day, at least 400 mg/day, at least 300 mg/day, at least 250 mg/day, at least about 200 mg/day, at least about 150 mg/day, and at least about 100 mg/day.

In a specific example, a daily dose of 1600 mg (R)-2-(2-fluoro-4-biphenyl)propionic acid (given as two 400 mg (R)-2-(2-fluoro-4-biphenyl)propionic acid tablets, BID) is administered to a patient. In another example, a daily dose of 800 mg (R)-2-(2-fluoro-4-biphenyl)propionic acid (given as two 400 mg (R)-2-(2-fluoro-4-biphenyl)propionic acid tablets, BID) is administered to a patient.

In a topical formulation, the compound may be present at a concentration of approximately 0.01 to 100 mg per gram of carrier.

In addition to administering the compound as a raw chemical, the compounds of the invention may be administered as part of a pharmaceutical preparation containing suitable pharmaceutically acceptable carriers comprising excipients and auxiliaries, which facilitate processing of the compounds into preparations that may be used pharmaceutically. For example, the preparations, particularly those preparations which may be administered orally and that may be used for the preferred type of administration, such as tablets, dragees, and capsules, and also preparations that may be administered rectally, such as suppositories, as well as suitable solutions for administration by injection or orally, may contain from approximately 0.01 to 99 percent, from approximately 0.25 to 75 percent of active compound(s), together with the excipient.

Also included within the scope of the present invention are the non-toxic pharmaceutically acceptable salts of the compounds of the present invention. Acid addition salts are formed by mixing a solution of the compounds of the present invention with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Basic salts are formed by mixing a solution of the compounds of the present invention with a solution of a pharmaceutically acceptable non-toxic base, such as sodium hydroxide, potassium hydroxide, choline hydroxide, sodium carbonate, Tris, N-methyl-glucamine and the like.

The pharmaceutical compositions of the invention may be administered to any animal, which may experience the beneficial effects of the compounds of the invention. Foremost among such animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner, which is itself known, e.g., by means of conventional mixing, granulating, dragee-making, dissolving, or lyophilizing processes. Thus, pharmaceutical preparations for oral use may be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular: fillers, such as saccharides, e.g. lactose or sucrose, mannitol or sorbitol; cellulose preparations and/or calcium phosphates, e.g. tricalcium phosphate or calcium hydrogen phosphate; as well as binders, such as starch paste, using, e.g., maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, e.g., silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropymethyl-cellulose phthalate, are used. Dye stuffs or pigments may be added to the tablets or dragee coatings, e.g., for identification or in order to characterize combinations of active compound doses.

Other pharmaceutical preparations, which may be used orally include push-fit capsules made of gelatin, as well as soft, sealed capsules made of gelatin and a plasticizer, such as glycerol or sorbitol. The push-fit capsules may contain the active compounds in the form of: granules, which may be mixed with fillers, such as lactose; binders, such as starches; and/or lubricants, such as talc or magnesium stearate and, optionally, stabilizers. In soft capsules, the active compounds may be dissolved or suspended in suitable liquids, such as fatty oils, or liquid paraffin. In addition, stabilizers may be added.

Possible pharmaceutical preparations, which may be used rectally include, e.g., suppositories, which consist of a combination of one or more of the active compounds with a suppository base. Suitable suppository bases are, e.g., natural or synthetic triglycerides, or paraffin hydrocarbons. In addition, it is also possible to use gelatin rectal capsules, which consist of a combination of the active compounds with a base. Possible base materials include, e.g., liquid triglycerides, polyethylene glycols, or paraffin hydrocarbons.

Suitable formulations for parenteral administration include aqueous solutions of the active compounds in water-soluble form, e.g., water-soluble salts and alkaline solutions. In addition, suspensions of the active compounds as appropriate oily injection suspensions may be administered. Suitable lipophilic solvents or vehicles include fatty oils, e.g., sesame oil, or synthetic fatty acid esters, e.g., ethyl oleate or triglycerides or polyethylene glycol-400 (the compounds are soluble in PEG-400), or cremophor, or cyclodextrins. Aqueous injection suspensions may contain substances which increase the viscosity of the suspension include, e.g., sodium carboxymethyl cellulose, sorbitol, and/or dextran. Optionally, the suspension may also contain stabilizers.

The topical compositions of this invention may be formulated as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included, as well as agents imparting color or fragrance, if desired. Additionally, transdermal penetration enhancers may be employed in these topical formulations. Examples of such enhancers are found in U.S. Pat. Nos. 3,989,816 and 4,444,762.

Creams may be formulated from a mixture of mineral oil, self-emulsifying beeswax and water in which mixture of the active ingredient, dissolved in a small amount of an oil, such as almond oil, is admixed. A typical example of such a cream is one which includes approximately 40 parts water, approximately 20 parts beeswax, approximately 40 parts mineral oil and approximately 1 part almond oil.

Ointments may be formulated by mixing a solution of the active ingredient in a vegetable oil, such as almond oil, with warm soft paraffin and allowing the mixture to cool. A typical example of such an ointment is one which includes approximately 30% almond oil and approximately 70% white soft paraffin by weight.

The following examples are illustrative, but not limiting, of the methods and compositions of the present invention. Other suitable modifications and adaptations of the variety of conditions and parameters normally encountered in clinical therapy and which are obvious to those skilled in the art are within the spirit and scope of the invention.

### EXAMPLE 1

### Identification of (R)-2-(2-fluoro-4-biphenyl)propionic acid as Agents for Treating Overactive Bladder and Urinary Incontinence

This Example provides a randomized, double-blind, placebo-controlled study of the effect of daily treatment with (R)-2-(2-fluoro-4-biphenyl)propionic acid. Study subjects have mild to moderate dementia of the Alzheimer's type and may be taking acetylcholinesterase (AChE) inhibitors provided the dose has been stable for at least 3 months. Subjects will be stratified at randomization for use/non-use of AChE inhibitors. A target of 201 subjects (67 subjects per arm) in 3 treatment groups are enrolled for 12 Months with optional follow-on treatment after Month 12 (2 treatment groups).

The study subjects are randomly divided into 3 groups for which the dosing regimen is one of following:
Group 1: 800 mg (R)-2-(2-fluoro-4-biphenyl)propionic acid (given as one 400 mg (R)-2-(2-fluoro-4-biphenyl)propionic acid tablet and 1 placebo tablet, BID);
Group 2: 1600 mg (R)-2-(2-fluoro-4-biphenyl)propionic acid (given as two 400 mg (R)-2-(2-fluoro-4-biphenyl)propionic acid tablets, BID); and
Group 3: Placebo (given as two placebo tablets, BID).

The intraday dosing interval is approximately 12 hours. Study drug is instructed to be taken at approximately the same time each day during the participation in the 12-month study. Study medication may be taken with or without food.

Proscribed therapy during the study period includes:
Initiation of, or change in dosage of, AChE inhibitors.
Treatment with memantine.
More than 7 days of NSAID use or aspirin >325 mg/day per month, including COX-2 specific inhibitors. (Use of cardioprotective doses of aspirin ≦325 mg/day is allowed.)
CYP2C9 substrates and inhibitors.
Ansaid®, Froben® or any other flurbiprofen-containing medication.
Other investigational medication or devices.
Cytotoxic chemotherapy.

A complete physical examination is performed by a medically qualified professional at screening and at Month 12 or Early Termination Prior to Month 12, and Month 24 (or End of Study). A review of all major body systems, including skin, head/ears/eyes/nose/throat (HEENT), respiratory, cardiovascular, gastrointestinal, endocrine/metabolic, genitourinary (if clinically relevant), neurological, blood/lymphatic, and musculoskeletal systems, is performed. Assessments of height (height is measured only at the Screening Visit), weight, and vital signs (systolic and diastolic blood pressure, pulse, temperature, and respirations) are included. All complete physical examination data is on the appropriate source documents.

A brief physical examination is performed by a medically qualified professional at Months 1, 3, 6, 9, 15, 18, 21 and 30-Day Off-Drug Follow-up. A review of body systems is assessed as appropriate evaluating and documenting any changes from previous visit. Assessment of vital signs (systolic and diastolic blood pressure, pulse, temperature, and respirations) are included. Review of laboratory results is evaluated for changes. Clinically significant changes are followed up per standard of care practice.

A standard 12-lead resting electrocardiogram (ECG) is performed at screening and at Month 12 or Early Termination Prior to Month 12, and Month 24 (or End of Study). It is preferred to have the Month 12 or Early Termination prior to Month 12 ECG conducted prior to venipuncture. The ECG readings and, if available, the computer analysis, are reviewed locally by an Investigator. The ECG report is reviewed, signed, and dated by the Investigator. Patients with clinically significant ECG findings are referred for follow-up as deemed appropriate by the Investigator.

The data were analyzed and the results showed that 6 patients in the placebo group exhibited urinary incontinence, 0 patients in the 400 mg BID group exhibited urinary incontinence (p value for 400 mg BID v. placebo=0.028) and 1 patient in the 800 mg BID group exhibited urinary incontinence (p value for 800 mg BID vs. placebo=0.063).

### EXAMPLE 2

### Acetic Acid Model for Evaluating Compounds in the Treatment of Overactive Bladder and Urinary Incontinence

Female rats (250-275 g BW) are anesthetized with urethane (1.2 g/kg) and a saline-filled jugular catheter (PE-50) is inserted for intravenous drug administration and a heparinized (100 units/ml) saline-filled carotid catheter (PE-50) is inserted for blood pressure monitoring. Via a midline abdominal incision from xyphoid to navel, a PE-50 catheter is inserted into the bladder dome for bladder filling and pressure recording.

The abdominal cavity of the animal is moistened with saline and closed by covering with a thin plastic sheet in order to maintain access to the bladder for filling cystometry emptying purposes. Fine silver or stainless steel wire electrodes are inserted into the external urethral sphincter (EUS) percutaneously for electromyography (EMG).

Saline and all subsequent infusates are continuously infused at a rate of about 0.055 ml/min via the bladder filling catheter for 30-60 minutes to obtain a baseline of lower urinary tract activity (continuous cystometry; CMG). Bladder pressure traces act as direct measures of bladder and urethral outlet activity, and EUS-E: H1G phasic firing and voiding act as indirect measures of lower urinary tract activity during continuous transvesical cystometry. Following the control period, a 0.25% acetic acid solution in saline (AA) is infused into the bladder to induce bladder irritation. Following 30 minutes of AA infusion, 3 vehicle injections are made at 20 minute intervals to determine vehicle effects, if any. Subsequently, increasing doses of a selected active agent are administered intravenously at 30 minute intervals in order to construct a cumulative dose-response relationship. At the end of the control saline cystometry period, the third vehicle injection, and 20 minutes following each subsequent treatment, the infusion pump is stopped, the bladder is emptied by fluid withdrawal via the infusion catheter and a single filling cystometrogram is performed at the same flow rate in order to determine changes in bladder capacity caused by the irritation protocol and subsequent drug administration.

### EXAMPLE 3

### Aβ Secretion Assay

To test whether compounds and compositions are capable of modulating Aβ levels, H4 neuroglioma cells expressing APP695NL and CHO cells stably expressing wild-type human APP751 and human mutant presenilin 1 (PS1) M146L are used. Generation and culture of these cells have been described. See Murphy et al., J. Biol. Chem., 274(17):11914-11923 (1999); Murphy et al., J. Biol. Chem., 275(34):26277-26284 (2000). To minimize toxic effects of the compositions and compounds, the H4 cells are incubated for 6 hours in the presence of the various compositions and compounds. To evaluate the potential for toxic effects of the compositions and compounds, additional aliquots of cells are incubated in parallel with each composition or compound. The supernatants are analyzed for the presence of lactate dehydrogenase (LDH) as a measure of cellular toxicity.

After incubating the cells with the compositions and compounds for a pre-determined time period, sandwich enzyme-linked immunosorbent assay (ELISA) is employed to measure secreted Aβ (Aβ42 and/or Aβ40) levels as described previously. Murphy et al., J. Biol. Chem., 275(34):26277-26284 (2000). For cell culture studies serum free media samples are collected following 6-12 hours of conditioning, Complete Protease Inhibitor Cocktail added (PIC; Roche), and total Aβ concentration measured by 3160/BA27 sandwich ELISA for Aβ₄₀ and 3160/BC05 sandwich ELISA for Aβ₄₂. All measurements are performed in triplicate. Antibody 3160 is an affinity purified polyclonal antibody raised against Aβ1-40. HRP conjugated monoclonal antibodies BA27 for detection of Aβ₄₀ and BC05 for detection of Aβ₄₂ have been previously described. Suzuki et al., Science, 264(5163):1336-1340 (1994).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A method of treating or slowing the onset of overactive bladder or urinary incontinence, or a symptom thereof selected from urinary frequency, urinary urgency, nocturia, or enuresis, comprising identifying and administering to a subject in need of treatment a therapeutically effective amount of an Aβ₄₂ lowering agent.

2. The method of claim 1, wherein the Aβ₄₂ lowering agent is a compound according to Formulae I-V: 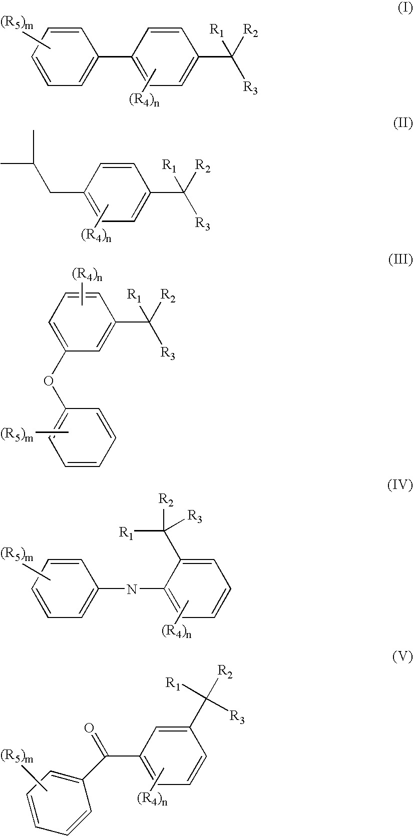
or pharmaceutically acceptable salts or solvates thereof, wherein:
R₁ is chosen from —CH₃, —CH₂CH₃, —CH₂CH₂CH₃, and —CH₂CH₂CH₂CH₃ (or can be taken together with R₂ to give a cyclopropyl ring, a cyclobutyl ring, a cyclopentyl ring, or a cyclohexyl ring);
R₂ is chosen from —CH₃, —CH₂CH₃, —CH₂CH₂CH₃, and —CH₂CH₂CH₂CH₃, (or can be taken together with R₁ to give a cyclopropyl ring, a cyclobutyl ring, a cyclopentyl ring, or a cyclohexyl ring);
R₃ is chosen from —COOH, —COOR₆, —CONH₂, —CONHR₆, —CONR₆R₇, —CONHSO₂R₆, tetrazolyl, and a —COOH bioisostere;
R₄ is chosen from —Cl, —F, —Br, —I, —CF₃, —OCF₃, —SCF₃, —OCH₃, —OCH₂CH₃, —CN, —CH═CH₂, —CH₂OH, and —NO₂;
R₅ is chosen from —Cl, —F, —Br, —I, —CF₃, —OCF₃, —SCF₃, —OCH₃, —OCH₂CH₃, —CN, —CH═CH₂, —CH₂OH, and —NO₂;
R₆ is chosen from —CH₃, —CH₂CH₃, —CH₂CH₂CH₃, and —CH₂CH₂CH₂CH₃;
R₇ is chosen from —CH₃, —CH₂CH₃, —CH₂CH₂CH₃, and —CH₂CH₂CH₂CH₃;
m is an integer chosen from 0, 1, 2, and 3; and
n is an integer chosen from 0, 1, 2, and 3.

3. The method of claim 1 wherein the Aβ₄₂ lowering agent is chosen from:
2-methyl-2 (2-fluoro-4′-trifluoromethylbiphen-4-yl) propionic acid; 2-methyl-2 (2-fluoro-4′cyclohexyl biphen-4-yl) propionic acid; 1-(2-fluoro-4′-trifluoromethylbiphenyl-4-yl)cyclopropanecarboxylic acid; 1-(4′-cyclohexyl-2-fluorobiphenyl-4-yl)cyclopropanecarboxylic acid; 1-(4′-benzyloxy-2-fluorobiphenyl-4-yl)cyclopropanecarboxylic acid; 1-(2-fluoro-4′-isopropyloxybiphenyl-4-yl)cyclopropanecarboxylic acid; 1-(2-fluoro-3′-trifluoromethoxybiphenyl-4-yl)cyclopropanecarboxylic acid; 1-(2-fluoro-4′-trifluoromethoxybiphenyl-4-yl)cyclopropanecarboxylic acid; 1-(2-fluoro-3′-trifluoromethylbiphenyl-4-yl)cyclopropanecarboxylic acid; 1-(4′-cyclopentyl-2-fluorobiphenyl-4-yl)cyclopropanecarboxylic acid; 1-(4′-cycloheptyl-2-fluorobiphenyl-4-yl)cyclopropanecarboxylic acid; 1-(2′-cyclohexyl-2-fluorobiphenyl-4-yl)cyclopropanecarboxylic acid; 1-(2-fluoro-4′-hydroxybiphenyl-4-yl)cyclopropanecarboxylic acid; 1-[2-fluoro-4′-(tetrahydropyran-4-yloxy)biphenyl-4-yl]-cyclopropane-carboxylic acid; 1-(2,3′,4′-trifluorobiphenyl-4-yl)cyclopropanecarboxylic acid; 1-(3′,4′-dichloro-2-fluorobiphenyl-4-yl)cyclopropanecarboxylic acid; 1-(3′,5′-dichloro-2-fluorobiphenyl-4-yl)cyclopropanecarboxylic acid 1-(3′-chloro-2,4′-difluorobiphenyl-4-yl)cyclopropanecarboxylic acid; 1-(4-benzo[b]thiophen-3-yl-3-fluorophenyl)cyclopropanecarboxylic acid; 1-(2-fluoro-4′-prop-2-inyloxy-biphenyl-4-yl)-cyclopropanecarboxylic acid; 1-(4′-cyclohexyloxy-2-fluoro-biphenyl-4-yl)-cyclopropanecarboxylic acid; 1-[2-fluoro-4′-(tetrahydropyran-4-yl)-biphenyl-4-yl]-cyclopropanecarboxylic acid; 1-[2-fluoro-4′-(4-oxo-cyclohexyl)-biphenyl-4-yl]-cyclopropanecarboxylic acid; 2-(2“-fluoro-4-hydroxy-[1,1′:4′,1′]tert-phenyl-4″-yl)-cyclopropanecarboxylic acid; 1-[4′-(4,4-dimethylcyclohexyl)-2-fluoro[1,1′-biphenyl]-4-yl]-cyclopropane-carboxylic acid; 1-[2-fluoro-4′-[[4-(trifluoromethyl)benzoyl]amino][1,1′-biphenyl]-4-yl]-cyclopropanecarboxylic acid; 1-[2-fluoro-4′-[[4-(trifluoromethyl)cyclohexyl]oxy][1,1′-biphenyl]-4-yl]-cyclopropanecarboxylic acid; 1-[2-fluoro-4′-[(3,3,5,5-tetramethylcyclohexyl)oxy][1,1′-biphenyl]-4-yl]-cyclopropanecarboxylic acid; 1-[4′-[(4,4-dimethylcyclohexyl)oxy]-2-fluoro[1,1′-biphenyl]-4-yl]-cyclopropanecarboxylic acid; 1-(2,3′,4″-trifluoro[1,1′:4′,1″-tert-phenyl]-4-yl)-cyclopropanecarboxylic acid; 1-(2,2′,4″-trifluoro[1,1′:4′,1″-tert-phenyl]-4-yl)-cyclopropanecarboxylic acid; 1-(2,3′-difluoro-4″-hydroxy[1,1′:4′,1″-tert-phenyl]-4-yl)-cyclopropane-carboxylic acid; 1-(2,2′-difluoro-4″-hydroxy[1,1′:4′,1″-tert-phenyl]-4-yl)-cyclopropane-carboxylic acid; 2-(2-fluoro-3′,5′-bis(chloro)biphen-4-yl)propionic acid amide; 2-(2-fluoro-4′-trifluoromethylbiphen-4-yl)propionic acid; 2-(2-fluoro-3′-trifluoromethylbiphen-4-yl)propionic acid; 2-(2-fluoro-3′,5′-bis(trifluoromethyl)biphen-4-yl)propionic acid; 2-(4′-cyclohexyl-2-fluorobiphen-4-yl)propionic acid; 2-(2-Fluoro-1,1′-biphenyl-4-yl) -2-methylpropanoic acid; 2-Methyl-2-(3-phenoxy-phenyl)-propionic acid; 2-(4-Isobutyl-phenyl)-2-methyl-propionic acid; 2-(6-Chloro-9H-carbazol-2-yl)-2-methyl-propionic acid; 2-[1-(4-Chloro-benzoyl)-5-methoxy-2-methyl-1H-indol-3-yl]-2-methyl-propionic acid; and 5-[1-(2-Fluoro-biphenyl-4-yl)-1-methyl-ethyl]-2H-tetrazole.

4. The method of claim 1, wherein the Aβ₄₂ lowering agent is (R)-2-(2-fluoro-4-biphenyl)propionic acid.

5. The method of claim 1, further comprising administering to the subject in need of treatment one or more additional therapeutic agents chosen from:
antimuscarinic agents; a muscosal surface protectant; an antihistamine; an anticonvulsant; a muscle relaxant; a bladder antispasmodic; a tricyclic antidepressant; a nitric oxide donor; a β₃-adrenergic receptor agonist; a bradykinin receptor antagonist; a neurokinin receptor antagonist; a sodium channel modulator, an activity dependent sodium channel modulator, and a Cav2.2 subunit calcium channel modulator.

6. The method of claim 5 wherein the Aβ₄₂ lowering agent is (R)-2-(2-fluoro-4-biphenyl)propionic acid.

7. The method of claim 1 wherein overactive bladder is treated.

8. The method of claim 1 wherein urinary incontinence is treated.

9. The method of claim 7 wherein the Aβ₄₂ lowering agent is (R)-2-(2-fluoro-4-biphenyl)propionic acid.

10. The method of claim 8 wherein the Aβ₄₂ lowering agent is (R)-2-(2-fluoro-4-biphenyl)propionic acid.

11. A method of treating or slowing the onset of overactive bladder or urinary incontinence, or a symptom thereof selected from urinary frequency, urinary urgency, nocturia, or enuresis, comprising identifying and administering to a subject with Alzheimer's disease who is in need of such treatment a therapeutically effective amount of an Aβ₄₂ lowering agent.

12. The method of claim 11, wherein the Aβ₄₂ lowering agent is a compound according to Formulae I-V: 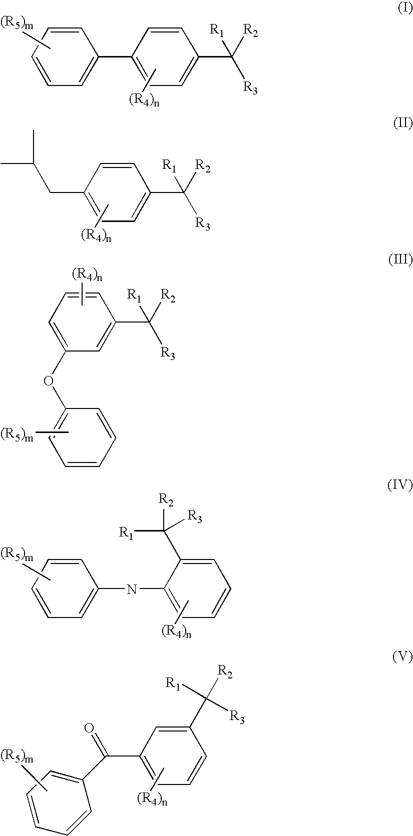
or pharmaceutically acceptable salts or solvates thereof, wherein:
R₁ is chosen from —CH₃, —CH₂CH₃, —CH₂CH₂CH₃, and —CH₂CH₂CH₂CH₃ (or can be taken together with R₂ to give a cyclopropyl ring, a cyclobutyl ring, a cyclopentyl ring, or a cyclohexyl ring);
R₂ is chosen from —CH₃, —CH₂CH₃, —CH₂CH₂CH₃, and —CH₂CH₂CH₂CH₃, (or can be taken together with R₁ to give a cyclopropyl ring, a cyclobutyl ring, a cyclopentyl ring, or a cyclohexyl ring);
R₃ is chosen from —COOH, —COOR₆, —CONH₂, —CONHR₆, —CONR₆R₇, —CONHSO₂R₆, tetrazolyl, and a —COOH bioisostere;
R₄ is chosen from —Cl, —F, —Br, —I, —CF₃, —OCF₃, —SCF₃, —OCH₃, —OCH₂CH₃, —CN, —CH═CH₂, —CH₂OH, and —NO₂;
R₅ is chosen from —Cl, —F, —Br, —I, —CF₃, —OCF₃, —SCF₃, —OCH₃, —OCH₂CH₃, —CN, —CH═CH₂, —CH₂OH, and —NO₂;
R₆ is chosen from —CH₃, —CH₂CH₃, —CH₂CH₂CH₃, and —CH₂CH₂CH₂CH₃;
R₇ is chosen from —CH₃, —CH₂CH₃, —CH₂CH₂CH₃, and —CH₂CH₂CH₂CH₃;
m is an integer chosen from 0, 1, 2, and 3; and
n is an integer chosen from 0, 1, 2, and 3.

13. The method of claim 11 wherein the Aβ₄₂ lowering agent is chosen from:
2-methyl-2(2-fluoro-4′-trifluoromethylbiphen-4-yl)propionic acid; 2-methyl-2(2-fluoro-4′cyclohexyl biphen-4-yl)propionic acid; 1-(2-fluoro-4′-trifluoromethylbiphenyl-4-yl)cyclopropanecarboxylic acid; 1-(4′-cyclohexyl-2-fluorobiphenyl-4-yl)cyclopropanecarboxylic acid; 1-(4′-benzyloxy-2-fluorobiphenyl-4-yl)cyclopropanecarboxylic acid; 1-(2-fluoro-4′-isopropyloxybiphenyl-4-yl)cyclopropanecarboxylic acid; 1-(2-fluoro-3′-trifluoromethoxybiphenyl-4-yl)cyclopropanecarboxylic acid; 1-(2-fluoro-4′-trifluoromethoxybiphenyl-4-yl)cyclopropanecarboxylic acid; 1 -(2-fluoro-3′-trifluoromethylbiphenyl-4-yl)cyclopropanecarboxylic acid; 1-(4′-cyclopentyl-2-fluorobiphenyl-4-yl)cyclopropanecarboxylic acid; 1-(4′-cycloheptyl-2-fluorobiphenyl-4-yl)cyclopropanecarboxylic acid; 1-(2′-cyclohexyl-2-fluorobiphenyl-4-yl)cyclopropanecarboxylic acid; 1-(2-fluoro-4′-hydroxybiphenyl-4-yl)cyclopropanecarboxylic acid; 1-[2-fluoro-4′-(tetrahydropyran-4-yloxy)biphenyl-4-yl]-cyclopropane-carboxylic acid; 1-(2,3′,4′-trifluorobiphenyl-4-yl)cyclopropanecarboxylic acid; 1-(3′,4′-dichloro-2-fluorobiphenyl-4-yl)cyclopropanecarboxylic acid; 1-(3′,5′-dichloro-2-fluorobiphenyl-4-yl)cyclopropanecarboxylic acid 1-(3′-chloro-2,4′-difluorobiphenyl-4-yl)cyclopropanecarboxylic acid; 1-(4-benzo[b]thiophen-3-yl-3-fluorophenyl)cyclopropanecarboxylic acid; 1-(2-fluoro-4′-prop-2-inyloxy-biphenyl-4-yl)-cyclopropanecarboxylic acid; 1-(4′-cyclohexyloxy-2-fluoro-biphenyl-4-yl)-cyclopropanecarboxylic acid; 1-[2-fluoro-4′-(tetrahydropyran-4-yl)-biphenyl-4-yl]-cyclopropanecarboxylic acid; 1-[2-fluoro-4′-(4-oxo-cyclohexyl)-biphenyl-4-yl]-cyclopropanecarboxylic acid; 2-(2“-fluoro-4-hydroxy-[1,1′:4′,1″]tert-phenyl-4″-yl)-cyclopropanecarboxylic acid; 1-[4′-(4,4-dimethylcyclohexyl)-2-fluoro[1,1′-biphenyl]-4-yl]-cyclopropane-carboxylic acid; 1-[2-fluoro-4′-[[4-(trifluoromethyl)benzoyl]amino][1,1′-biphenyl]-4-yl]-cyclopropanecarboxylic acid; 1-[2-fluoro-4′-[[4-(trifluoromethyl)cyclohexyl]oxy][1,1′-biphenyl]-4-yl]-cyclopropanecarboxylic acid; 1-[2-fluoro-4′-[(3,3,5,5-tetramethylcyclohexyl)oxy][1,1′-biphenyl]-4-yl]-cyclopropanecarboxylic acid; 1-[4′-[(4,4-dimethylcyclohexyl)oxy]-2-fluoro[1,1′-biphenyl]-4-yl]-cyclopropanecarboxylic acid; 1-(2,3′,4″-trifluoro[1,1′:4′,1″-tert-phenyl]-4-yl)-cyclopropanecarboxylic acid; 1-(2,2′,4″-trifluoro[1,1′:4′,1″-tert-phenyl]-4-yl)-cyclopropanecarboxylic acid; 1-(2,3′-difluoro-4″-hydroxy[1,1′: 4′,1″-tert-phenyl]-4-yl)-cyclopropane-carboxylic acid; 1-(2,2′-difluoro-4″-hydroxy[1,1′:4′,1″-tert-phenyl]-4-yl)-cyclopropane-carboxylic acid; 2-(2-fluoro-3′,5′-bis(chloro)biphen-4-yl)propionic acid amide; 2-(2-fluoro-4′-trifluoromethylbiphen-4-yl)propionic acid; 2-(2-fluoro-3′-trifluoromethylbiphen-4-yl)propionic acid; 2-(2-fluoro-3′,5′-bis(trifluoromethyl)biphen-4-yl)propionic acid; 2-(4′-cyclohexyl-2-fluorobiphen-4-yl)propionic acid; 2-(2-Fluoro-1,1′-biphenyl-4-yl) -2-methylpropanoic acid; 2-Methyl-2-(3-phenoxy-phenyl)-propionic acid; 2-(4-Isobutyl-phenyl)-2-methyl-propionic acid; 2-(6-Chloro-9H-carbazol-2-yl)-2-methyl-propionic acid; 2-[1-(4-Chloro-benzoyl)-5-methoxy-2-methyl-1H-indol-3-yl]-2-methyl-propionic acid; and 5-[1-(2-Fluoro-biphenyl-4-yl)-1-methyl-ethyl]-2H-tetrazole.

14. The method of claim 11, wherein the Aβ₄₂ lowering agent is (R)-2-(2-fluoro-4-biphenyl)propionic acid.

15. The method of claim 11, further comprising administering to the subject in need of treatment one or more additional therapeutic agents chosen from:
antimuscarinic agents; a muscosal surface protectant; an antihistamine; an anticonvulsant; a muscle relaxant; a bladder antispasmodic; a tricyclic antidepressant; a nitric oxide donor; a β₃-adrenergic receptor agonist; a bradykinin receptor antagonist; a neurokinin receptor antagonist; a sodium channel modulator, an activity dependent sodium channel modulator, and a Cav2.2 subunit calcium channel modulator.

16. The method of claim 15 wherein the Aβ₄₂ lowering agent is (R)-2-(2-fluoro-4-biphenyl)propionic acid.

17. The method of claim 11 wherein overactive bladder is treated.

18. The method of claim 11 wherein urinary incontinence is treated.

19. The method of claim 17 wherein the Aβ₄₂ lowering agent is (R)-2-(2-fluoro-4-biphenyl)propionic acid.

20. The method of claim 18 wherein the Aβ₄₂ lowering agent is (R)-2-(2-fluoro-4-biphenyl)propionic acid.

21. The method of claim 11, wherein the subject has mild to moderate Alzheimer's disease or mild cognitive impairment.

22. The method of claim 21, wherein the Aβ₄₂ lowering agent is (R)-2-(2-fluoro-4-biphenyl)propionic acid.

23. The method of claim 22, further comprising administering to the subject in need of treatment one or more additional therapeutic agents chosen from:
antimuscarinic agents; a muscosal surface protectant; an antihistamine; an anticonvulsant; a muscle relaxant; a bladder antispasmodic; a tricyclic antidepressant; a nitric oxide donor; a β₃-adrenergic receptor agonist; a bradykinin receptor antagonist; a neurokinin receptor antagonist; a sodium channel modulator, an activity dependent sodium channel modulator, and a Cav2.2 subunit calcium channel modulator.

24. The method of claim 22, wherein overactive bladder is treated.

25. The method of claim 22 wherein urinary incontinence is treated.
